(19) United States
(12) Patent Application Publication
(10) Pub. No.: US 2006/0240078 A1
(43) Pub. Date: Oct. 26, 2006

(54) Feed supplement delivery system

(75) Inventors: Thomas C. Jenkins, Central, SC (US); Karen J.L. Burg, Clemson, SC (US); Steve E. Ellis, Central, SC (US)

Correspondence Address: DORITY & MANNING, P.A., POST OFFICE BOX 1449, GREENVILLE, SC, 29602-1449, US

(73) Assignee: Clemson University

(21) Appl. No.: 11/389,668

(22) Filed: Mar. 24, 2006

Related U.S. Application Data
(60) Provisional application No. 60/664,742, filed on Mar. 24, 2005.

(51) Int. Cl. A61K 9/16; A23K 1/165; A61K 9/50

(52) U.S. Cl. 424/442; 424/490

(57) ABSTRACT

Disclosed are novel feed supplements for ruminants and methods for making the same. The feed supplements include unsaturated fatty acid encapsulated by a protective coating. Through utilization of the disclosed feed supplements, dietary intake and absorption of unsaturated fatty acids can be increased, due to protection of the ingested unsaturated fatty acids from biohydrogenation in the animal's rumen. The methods of the invention can be utilized to increase unsaturated fatty acid levels in the animal's tissues. Hence, food products obtained from the animal can also have an increased unsaturated fatty acid content and correspondingly lower saturated fatty acid content.

### PRIORITY INFORMATION

The present application claims priority to the provisional patent application having the Ser. No. 60/664,742 filed on Mar. 24, 2005, which is hereby incorporated by reference in its entirety.

### FEDERALLY SPONSORED RESEARCH AND DEVELOPMENT

The United States Government may have rights in this invention pursuant to National Science Foundation REU grants No. BES-0139624 and EEC-0139624.

### BACKGROUND OF THE INVENTION

Medical and nutritional specialists routinely advise consumers to limit their intake of saturated fatty acids. In response to this need, many food industries are attempting to limit the amount of saturated fatty acids in their food products. For instance, the dairy industry has developed many products varying widely in overall fat content.

In order to provide healthier dairy and meat products, the beef and dairy industries have used many high quality feed products which include unsaturated fatty acids designed to produce healthier and larger cows and healthier food products from the cows. However, most of the unsaturated fatty acids in the feed products are converted to saturated fatty acids during digestion. For instance, the microbial population of the rumen can convert most of the unsaturated fatty acid consumed by a ruminant into saturated fatty acid through biohydrogenation, directly affecting the amount of the unsaturated fatty acid that can be absorbed into the ruminant's bloodstream. One of the results of biohydrogenation is that the unsaturated fatty acid content within meat and dairy products supplied by ruminants is relatively low compared to the saturated fatty acid content, making those products less healthy for the consumer.

Many attempts have been made to increase the amount of unsaturated fatty acid surviving biohydrogenation and eventually being absorbed into a ruminant's bloodstream. However, these attempts have met with little practical success. For instance, probably the most widely known fat developed to resist biohydrogenation in order to increase polyunsaturated fatty acid levels in milk was the formaldehyde-treated lipid. Feeding formaldehyde-treated lipids to cattle and sheep was shown to significantly elevate polyunsaturated fatty acids levels in tissues of cattle and sheep. Furthermore, milk unsaturated fatty acids also increased when formaldehyde-treated lipid was fed to lactating cows. However, commercial application of formaldehyde-treated lipid was never achieved in the United States, most likely due in large part to health risks associated with the use of formaldehyde.

Another attempt to increase the amount of unsaturated fatty acid absorbed during digestion has involved feeding cows whole oilseeds, such as soybeans, cottonseeds, and sunflower seeds, resulting in increase of unsaturated fatty acids in tissue and milk composition according to some reports. Whole seeds provide some protection from biohydrogenation due to the nature of their hard outer seed coat. However, a disruption of the seed coat exposes the oil to the microbial population, increasing the potential for biohydrogenation. Since the seed coat can be sufficiently disrupted by ordinary chewing, the whole oilseeds do not provide a practical way to consistently increase the amount of unsaturated fatty acids that avoid biohydrogenation.

Another attempt at increasing the unsaturated fatty acid uptake of ruminants involved feeding calcium salts of fatty acids to animals. However, it appeared that unsaturated fatty acids were only protected from hydrogenation in the rumen when encapsulated inside an insoluble matrix of saturated calcium salts. Thus, the desired protection was only possible when unsaturated fatty acid content was low, which greatly reduced the extent that unsaturated fatty acid content could be altered in the meat or milk of the ruminant.

There exists a need for methods and products directed to increasing the amount of unsaturated fatty acid absorbed into the blood supply of ruminants. In particular, there exists a need for methods and products that can increase the overall health of a ruminant and/or increase the unsaturated fatty acid content of food products supplied by the ruminant.

### SUMMARY OF THE INVENTION

Generally speaking, the present invention is directed to a feed supplement for protecting unsaturated fatty acid from hydrogenation. The feed supplement can comprise an unsaturated fatty acid and a protective coating. The protective coating encapsulates the unsaturated fatty acid. The protective coating comprising a biocompatible polymer.

In one embodiment, at least 50% of the unsaturated fatty acid encapsulated by the protective coating can remain unhydrogenated after the feed supplement is exposed to ruminal fluid for at least about 24 hours.

For example, the biocompatible polymer can be biodegradable through hydrolysis, such as enzymatic hydrolysis or non-enzymatic hydrolysis. In one embodiment, the bio-compatible polymer can be a biocompatible aliphatic polyester, such as a polyhydroxy acid. For instance, the biocompatible polymer can be a lactide-based polymer.

The present invention is also generally directed to a method preparing a feed supplement for ruminants. The method comprises encapsulating unsaturated fatty acid with a protective coating. The protective coating comprises a biocompatible aliphatic polyester polymer. At least 50% of said unsaturated fatty acid encapsulated by the protective coating can remain unhydrogenated after the feed supplement is exposed to ruminal fluid for at least about 24 hours.

### DETAILED DESCRIPTION OF THE INVENTION

Reference now will be made in detail to various embodiments of the invention, one or more examples of which are set forth below. Each example is provided by way of explanation of the invention, not limitation of the invention. In fact, it will be apparent to those skilled in the art that various modifications and variations can be made in the present invention without departing from the scope or spirit of the invention. For instance, features illustrated or described as part of one embodiment, can be used on another embodiment to yield a still further embodiment. Thus, it is intended that the present invention covers such modifications and variations as come within the scope of the appended claims and their equivalents.

In general, the present invention is directed to methods and products that can protect unsaturated fatty acid (UFA) from hydrogenation to saturated fatty acids (SFA) during digestion by a ruminant. For example, in one embodiment, the disclosed products and methods can be utilized to protect ingested UFA from hydrogenation to SFA prior to absorption, and thus can promote the absorption of increased levels of UFA into the animal's bloodstream during digestion. Increasing the amount of UFA absorbed during digestion can result in better health of the ruminant as well as increased UFA content and decreased SFA content of food products supplied by the ruminant.

In one embodiment, the disclosed invention can be utilized to more closely control the ratio of SFA to UFA absorbed by the animals. For example, the ratio of SFA to UFA can be controlled in a dairy cow so as to control hardness of the fat obtained from the animal. In general, the ratio of fatty acids with high melting point (generally more saturated) to those with low melting points (generally more unsaturated) determine whether the fat will be hard or soft. For instance, soft fat has a high content of low-melting fatty acids and at room temperature has a large continuous fat phase with a low solid phase (crystallized, high-melting fat). On the other hand, a hard fat has a larger content of the solid phase of high-melting fat than the continuous fat phase of low-melting fatty acids. Processing technologies that can alter milk fatty acid composition and SFA:UFA ratio in dairy products are currently being examined by the dairy industry but are hampered by high cost along with complicated, lengthy procedures.

According to one embodiment of the invention, the hardness of the fat of an animal can be increased or decreased, depending on the desired characteristics of the end product that incorporates the fat. For example, in one embodiment, the SFA:UFA ratio can be decreased by increasing the uptake of UFA. As such, the animal can produce softer milk fat, which can in turn be utilized to produce, for instance, softer, more “spreadable” butter.

In another embodiment, through the methods of the present invention the total UFA uptake by the animal can be lowered somewhat as compared to the previous embodiment, with a corresponding increase in SFA uptake. Thus, the SFA:UFA ratio can be increased and the hardness of the animal's milk fat can also increase. An increase in fat hardness can provide for, e.g., the production of cheeses that are more easily grated.

Another possible benefit of controlling the UFA uptake of a ruminant and in particular, of increasing the amount of UFA absorbed by the animal during digestion, can be improvements in reproductive performance. For instance, studies have shown that merely feeding UFA to lactating dairy cows can improve reproductive performance, implying possible benefits on lifetime production potential of the cow. The reported reproductive improvements include higher conception rates, increased pregnancy rates, and reduced open days.

In another embodiment, the present invention is directed to an economical, relatively simple process for the production of food products, for instance dairy and meat products, having increased UFA content. In particular, by increasing the proportion of UFA absorbed during digestion according to the present invention, a ruminant can produce food products having a fatty acid composition with a higher UFA content and lower SFA content. Diets high in SFA content have been linked to high cholesterol levels and greater risk of cardiovascular disease in both humans and ruminants. On the other hand, increasing UFA intake has been shown to lower blood cholesterol levels.

The present invention is generally directed to fatty acid digestion in ruminants. Ruminants are a class of animals, such as cows, goats, deer, moose and sheep, distinguished by their multi-compartment stomachs. Typically, ruminants have 4 major stomach compartments: the rumen, the reticulum, the omasum, and the abomasum. During the digestive process, the feed passes first to the rumen, the largest compartment of the digestive system. The rumen of mature cattle describes an aqueous environment of roughly a 40-50 gallon capacity that supports a microbial population including bacteria, fungi, and protists at a relatively constant temperature and pH. The microbial population of the rumen is responsible for fermentation and transformation of dietary lipid that enables the animal to survive and thrive on fodder that is indigestible by other animals.

Among other functions, the microbial population of the rumen is responsible for lipolysis and biohydrogenation of the fiber found in the animal's feed such as hay, silage and pasture. The primary waste product of the biohydrogenation process by the microbial population in the rumen is SFA. For instance, both monounsaturated and polyunsaturated fatty acids either existing in the feed as such or formed during the initial digestive processes of the feed by the microbes, can be converted into saturated fatty acid during biohydrogenation.

For example, linoleic acid is a common UFA found in animal feed. Biohydrogenation of linoleic acid in the rumen can begin with the conversion of linoleic acid to conjugated linoleic acid (CLA), in which the total number of double bonds on the backbone of the carbon chain remains the same but one of the double bonds is shifted to a new position by microbial enzymes. Many types of CLA are produced in the rumen of dairy cows, but a common CLA produced from biohydrogenation of linoleic acid is cis-9, trans-11 C18:2. As the biohydrogenation progresses, double bonds in the CLA intermediates can then be hydrogenated further to trans fatty acids having only one double bond. A final hydrogenation step by the ruminal microbes can eliminate the last double bond yielding the SFA stearic acid as the final end product. Waste products of the microbial processing such as stearic acid can begin to be absorbed by the animal through the lining of the rumen wall or can be passed to the rest of the digestive system with the remaining feed that is not subject to biohydrogenation.

Biohydrogenation can greatly reduce the quantity of dietary UFA available for uptake into the bloodstream during digestion. For example, intake of linoleic acid by dairy cows typically ranges from about 70 to about 200 g/day, but only about 10 to about 50 grams of ingested linoleic acid usually survive biohydrogenation to reach the small intestine based on this diet. In contrast, about 500 g of saturated stearic acid can reach the small intestine of a dairy cow each day, even though only few grams of stearic acid are consumed. Typically, stearic acid can be the primary fatty acid absorbed by cows regardless of the quantity of UFA consumed in the diet.

After processing in the rumen, remaining undigested feed and unabsorbed biohydrogenation products pass through the omasum and the abomasum, and into the intestines where enzymatic break down of proteins into peptides and amino acids, starches into glucose, and complex fats into fatty acids can be carried out. The molecular products of enzymatic digestion as well as the remaining biohydrogenation products of microbial digestion can then be absorbed into the blood stream of the animal or excreted as waste. Importantly, however, conversion of UFA to SFA will primarily only occur during the biohydrogenation processes of the rumen, and UFA that survives digestion by the microbial population of the rumen can be absorbed as such, as the additional enzymatic digestion processes of the remaining digestive system will generally not convert UFA to SFA.

According to the present invention, a feed supplement can be provided that can encapsulate UFA within a protective coating. As used herein, the term “encapsulate” is meant to include any manner of containing a material within the boundaries of the coating. As such, a protective coating “encapsulating” unsaturated fatty acid can be a continuous coating or a porous coating (such as a mesh coating), as long as the unsaturated fatty acid is contained and substantially held within the protective coating.

In general, the protective coating of the present invention can be designed so as to survive the environment of the rumen with little or no degradation and thus protect the material held inside from biohydrogenation. For example, the protective coating can degrade less than about 20% by weight after exposure to ruminal fluid for 24 hours, such as less than about 15% by weight. For example, in one particular embodiment, the protective coating can degrade less than about 10% by weight after exposure to ruminal fluid for 24 hours, such as less than about 5% by weight.

As the digestive process continues, however, the protective coating can degrade in other areas of the ruminant's digestive system, and the material held inside the coating can then be released to be digested by the animal. For example, the supplement can resist degradation within the rumen such that at least some of the material encapsulated in the supplement can only be released from the protective coating in the abomasum or the intestines of the ruminant.

Thus, the protective coating can protect substantially all of the unsaturated fatty acid from biohydrogenation by the microbial population of the rumen. For example, greater than 50% of the unsaturated fatty acid can be protected from biohydrogenation in ruminal fluid when exposed for about 24 hours. For instance, greater than 60% of the unsaturated fatty acid can be protected from biohydrogenation in ruminal fluid when exposed for about 24 hours, such as greater than 75%. In one particular embodiment, greater than about 85% of the unsaturated fatty acid can be protected from biohydrogenation in ruminal fluid when exposed for about 24 hours, such as greater than 90% or greater than about 95%.

In general, the feed supplements of the present invention can be any size or shape capable of being ingested by a ruminant. For instance, in one embodiment, the feed supplements can be individual beads, chips, scaffolds, pellets, spheres, microspheres, or the like and can be fed to the animals as such. In one embodiment, the feed supplement can be included within another capsule for delivery to the ruminant. For instance, a plurality of microspheres can be contained within a capsule. In another embodiment, the feed supplements can be combined with other materials and be ingested by the animals in this form. For example, the feed supplements of the invention can be combined with other materials commonly fed to the animals, such as processed dry feed materials or salt and can be ingested by the animal in this combination form.

The protective coating can include one or more materials that are highly resistant to degradation in the environments of certain sections of the digestive system, but more susceptible to degradation in other areas. For example, the protective coating can include materials that are highly resistant to degradation in the anearobic, microbial-rich environment of the rumen, but susceptible to degradation in the acidic, aqueous environment of the abomasum. Following exit from the rumen with little or no degradation due to microbial digestive processes, the supplement can be subject to rapid degradation in the acidic abomasums or to enzymatic hydrolysis in the intestines. Then, the material (e.g. unsaturated fatty acid) held inside the protective coating can be released where it can be subject to enzymatic digestion or optionally immediately available for absorption into the animal's bloodstream.

The protective coating of the disclosed feed supplement may be formed of a single polymeric component or optionally may be a combination of multiple monomeric or polymeric components. For instance, the protective coating can, in one embodiment, be a polymeric coating formed of a single polymerized monomer, for instance either a natural or synthetic monomer that can be polymerized to form a polymeric encapsulation surrounding the protected material inside. In particular, the polymeric formation can be resistant to microbial digestion in the rumen and thus delay release of the protected material carried inside the encapsulation. For example, the coating can be resistant to microbial degradation due to the physical characteristics of the encapsulation, e.g., the thickness of the polymerized coating itself. Alternatively, the coating can resist microbial degradation due to the polymer used to form the coating.

For example, in one embodiment, the coating can include a biocompatible polymer. The biocompatible polymer can be degradable by hydrolysis, either enzymatic or non-enzymatic. For example, the biocompatible polymer can be a biocompatible aliphatic polyester, such as a polyhydroxyacid polymer.

In one particular embodiment, the biocompatible polymer can be a lactide-based polymer. For purposes of this disclosure, the term “lactide-based polymer” is intended to be synonymous with the terms polylactide, polylactic acid (PLA) and polylactide polymer, and is intended to include any polymer formed from the ring opening polymerization of lactide monomers, either alone (i.e., homopolymer) or in mixture or copolymer with other monomers. The term is also intended to encompass any different configuration and arrangement of the constituent monomers (such as syndiotactic, isotactic, and the like). For instance, the lactide-based polymer can be poly-L-lactide, which is naturally resistant to microbial degradation. Alternatively, the lactide-based polymer can be poly-D,L-lactide.

Lactic acid is produced commercially by fermentation of agricultural products such as whey, cornstarch, potatoes, molasses, and the like. When forming a lactide-based polymer, the lactide monomer can first be formed by the depolymerization of the lactic acid oligomer. In the past, production of lactide was a slow, expensive process, but recent advances in the art have enabled the production of high purity lactide at reasonable costs. As such processes are generally known to those of skill in the art; however, they are not discussed at length herein.

In one embodiment, the methods of the present invention can include formation of a lactide-based polymer through the ring-opening polymerization of a lactide monomer. Polymerization of the lactide monomer can occur in the presence of a suitable polymerization catalyst, generally at elevated heat and pressure conditions, as is generally known in the art. In general, the catalyst can be any compound or composition that is known to catalyze the polymerization of lactide. In other embodiments, commercially available polymers can be used.

The chiral carbon atom in the lactic acid structure results in the three stereoisomers of lactide, shown below:

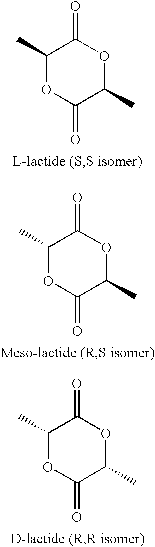

According to the processes of the present invention, either racemic mixtures or pure enantiomers of lactide may be utilized. In general, a polymer of only L-lactide monomers may be preferred; however, a polymer of L- and D-lactide monomers may be utilized due to economic realities, though this is not a requirement of the invention.

In one embodiment, the protective coating can include two or more components in some combination. This embodiment can provide additional means to control the degradation rate of the protective coating, such as to, for example, better ensure suitable degradation of the coating to provide release of the contents for absorption by the animal prior to excretion as waste. For example, in one embodiment, two or more individual monomeric components can be polymerized together to form a single coating layer of a copolymer. For example, the copolymer can be a lactide-based polymer, such as polylactide-co-caprolactone. In another embodiment, two or more polymers can be combined to form a layer comprising a block-copolymer having the desired degradation characteristics. According to this embodiment, the physical and/or chemical characteristics of the coating layer can be varied so as to control the degradation rate of the protective coating. For example, purely physical characteristics, such as thickness of a bi-component coating layer can be varied to alter the degradation rate of the layer, as described above for a single component system.

However, due to the combination of two or more different components, additional degradation rate control mechanisms can be provided. For example, in one embodiment two or more components can be combined that display a different susceptibility to microbial and/or enzymatic degradation. Accordingly, the overall degradation rate of the protective coating can be varied by variance of, for example, the relative percentage of each component in the protective coating or the molecular weight of each component used in forming the coating. For example, polylactide-co-caprolactone can be made with a ratio of lactide to caprolactone of from about 50:50 to about 90:10, such as about 75:25.

In one embodiment, the polymeric encapsulation can include additional agents, such as cross-linking agents, for example, that can increase the material's resistance to microbial degradation. However, because of the nature of many cross-linking agents, they may not be biocompatible. As such, in one particular embodiment, the protective coating can be free of cross-linking agents.

Optionally, particular characteristics of the polymer used to form the coating can be varied, such as molecular weight of the polymerized monomer, for example, so as to vary the degradation rate of the polymeric coating. Typically, the polymer of the protective coating will have an average molecular weight of from about 20,000 Da to about 1,000,000 Da. In most applications, however, the average molecular weight of the polymer in the protective coating will be lower (i.e. about 20,000 to about 100,000) to aid in the processing and application of the coating to the unsaturated fatty acid.

In another embodiment, the crystallinity of the coating can be varied, which can alter the rate of the microbial digestion of the material. Typically, the polymer of the protective coating will be amorphous to semi-crystalline, such as having a crystallinity of from 0% to about 40%.

Importantly, however, the coating must be such that it can eventually degrade, to at least some degree, in the animal's digestive system to release the protected materials. For example, if the coating is highly resistant to microbial digestion, it should present less resistance to either enzymatic digestive processes or the environment of later sections of the digestive system, such as the acidic environment of the abomasum, for example, so as to cause release of the contents.

For example, the protective coating can substantially survive past the abomasums and be broken down by the protein degrading enzymes that are secreted in the first part of the small intestines to digest protein.

In general, the protective coating of the disclosed feed supplement can include either a single layer or multiple layers. Moreover, each layer can be the same or different from adjacent layers. For instance, in one embodiment, the protective coating may comprise a first inner layer that can completely surround the encapsulated material, and a second layer that can be the same or different composition as the first layer that can substantially cover the first layer, and so on with additional layers. In addition, adjacent layers can differ from one another in any way. For example, adjacent layers can be formed of completely different materials. In one embodiment, adjacent layers can share one or more components. For example, a first layer can include a monomeric or polymeric component in common with a second, adjacent layer, but the first and/or the second layer can also include additional components. In one embodiment, adjacent layers can be formed of essentially the same components, but the layers can differ in proportion of the common components.

In one embodiment, the protective coating can include a layer that is not continuous across the entire surface of another layer of the protective coating. For example, following formation of a substantially complete first layer encapsulating a protected material, a second discontinuous layer can be formed on the first layer of the same or different components that can only partially cover the first layer, as in patches or only in a single area. The presence of the second, discontinuous layer can contribute to the degradation rate control of the supplement during digestion such as by, for example, limiting the surface area of the first layer that can be accessible to the microbial population of the rumen.

The components that can be utilized in forming the feed supplements of the present invention can be either monomeric or polymeric components. In general, the components can be biocompatible materials. In addition, at least one component used in forming the protective coatings can be susceptible to at least one of microbial or enzymatic digestive processes in a ruminant.

For example, in one embodiment, the protective coatings of the present invention can include natural polymers such as, but not limited to, alginate, gelatin, proteins, albumin, synthetic polyamino acids, prolaminescollagen, polysaccharide, gelatin, fibrin, hyaluronic acid, agar, agarose, gum arabic, fibronectin, laminin, glycosaminoglycan, polyvinyl alcohol, and any mixture or combination thereof.

In another exemplary embodiment, synthetic monomeric or polymeric components can be utilized. For example, in one embodiment, biocompatible aliphatic polyesters, such as lactide-based polymers can be utilized. A non-limiting list of synthetic polymeric materials that can be utilized in forming the disclosed protective coatings can include polylactides, (e.g. poly-L-lactide (PLLA) and poly-D,L-lactide (PDL)), polylactide-co-caprolactone (PLLA/PLC), aliphatic polyesters, poly(amino acids), copoly(ether-esters), polyalkylene oxalates, polyamides, poly(iminocarbonates), polyacetals, polycyanoacrylates, degradable polyurethanes, polyorthoesters, polyoxaesters, polyamidoesters, polyoxaesters comprising amine groups, poly(anhydrides), polyphosphazenes, polysaccharides, polyacrylates, poly(vinyl amines), biopolymers, and any mixtures or combination thereof.

In one embodiment, polymeric components utilized in forming the protective coatings of the invention can be thermoplastic materials, but it should be understood that this is not a requirement of the invention. For example, in some embodiments, the protective coatings can utilize various thermoset materials.

In addition, it should be understood that the disclosed protective coatings can, in certain embodiments, include non-degradable components. For instance, in one embodiment, a component of a multi-component system can be a non-degradable or non-digestible material that can be excreted by the animal. In this embodiment, however, the protective coating must also include a degradable portion in some combination with the non-degradable portion so as to allow the materials held within the protective coating to be released upon at least partial degradation of the degradable portion of the protective coating.

The protective coating can have any thickness capable of achieving the desired protection of the encapsulated unsaturated fatty acid. For example, the thickness of the protective coating can be from a discontinuous coat (e.g. a mesh coating) to about 1 millimeter. For instance, the protective coating can be a continuous coating having a thickness from about 0.1 millimeter to about 0.5 millimeters.

In one embodiment, the protective coating of the feed supplement can include one or more materials that can be susceptible to the microbial digestion of the rumen, but the rate of the digestion of the coating can be controlled so as to protect the encapsulated contents. For instance, materials can be utilized in forming the protective coating that can be somewhat susceptible to degradation in the microbial-rich environment of the rumen, but this susceptibility can be controlled through some characteristic of the coating (e.g., material characteristics such as thickness, chemical structure, cross-link density, etc.) so as to slow the degradation rate of the coating. As such, the protective coating of the feed supplement can protect the materials held within the supplement by use of a predetermined degradation rate of the protective coating. For example, the degradation rate of the protective coating can be such that a majority of the protective coating can be expected to survive the microbial degradation of the rumen, though a certain amount of degradation of the coating can occur through this section of the digestive system. As the protective coating degrades over time, the material held within the supplement can begin to be released. However, due to the time delay in release, at least some of the material can be released into digestive system after the supplement has passed through the rumen, and thus, this material can be protected from biohydrogenation by the microbial population.

In one embodiment, UFA can be directly encapsulated as such within the protective coating of the disclosed feed supplements. Generally, a long chain fatty acid, having up to 30 carbons in length and having anywhere from 1 to about 6 double bonds, can be included in the enclosed feed supplements. For example, the unsaturated fatty acid can be from about 10 carbons to about 26 carbons in length.

For example, fatty acids might include any of the omega 3 fatty acids or any of the omega 6 fatty acids and could also include trans-fatty acids, such as found in conjugated linoleic acids. For instance, unsaturated fatty acids including, but not limited to, oleic acid (C18:1), palmitoleic acid (C16:1), vaccenic acid (C18:1), linoleic acid (C18:2), conjugated linoleic acid (C18:2), linoleic acid (C18:3), arachidonic acid (C20:4), eicosapentaenoic acid (C20:5), and/or docosahexaenoic acid (C22:6) can be encapsulated within the protective coatings either individually or in combination.

In one embodiment, other bioactive substances can be encapsulated within the protective coating and protected from the microbial population of the rumen. For example, vitamins and pharmaceuticals can be encapsulated within the protective coating. Also, more complex materials can be encapsulated within the protective coatings and protected from microbial digestion according to the present invention. For example, in one embodiment, complex fats such as esterfied lipids (triacylglycerols, phospholipids, and the like) could be encapsulated in the protective coatings. Following suitable degradation of the coatings, the fats can be released from the supplements and subjected to enzymatic digestion to form UFA that can then be absorbed by the animal. According to this embodiment, materials that are physically larger than the readily absorbable UFA can be encapsulated within the protective coatings. As such, more substantial degradation of the protective coatings can be required prior to release of the encapsulated materials as compared to the embodiment described above, wherein small UFA are encapsulated. As such, the expected time lag between the time of ingestion of the supplements and the release of larger, more complex contents of the supplements into the digestive system can be longer in this particular embodiment than in embodiments wherein a similar coating material is used to encapsulate UFA.

Formation of the disclosed feed supplements can be performed through application of or formation of the protective coating material so as to encapsulate the materials held within the protective coatings according to any encapsulation method as is generally known to one skilled in the art. For example, the disclosed feed supplements can be formed by processing techniques generally known in the art including compression molding, extrusion, dip coating, modified emulsion/evaporation techniques, spray coating, solvent casting/particulate leaching methods and the like. In addition, any combination of these or other known methods can also be employed to form the disclosed feed supplements.

### EXAMPLES

### Example 1

Three-dimensional, porous scaffolds were fabricated via a solvent casting/particulate leaching method, as disclosed in Webster, S. S., et al., J. Histotechnol 2003, 26(1):57-65, incorporated by reference herein, of both poly-D,L-lactide (PDL) and poly-L-lactide-co-caprolactone (PLLA/PLC), without the inclusion of any fatty acids. Briefly, the polymer of interest was dissolved in chloroform to form a 0.05 g/ml solution. Sigmacote was then applied to casting containers (standard 10mi glass beakers) and 1 ml of porogen (in this case NaCl) was added to the bottom of the containers. The polymer solution was then added to the containers and the solvent was allowed to evaporate at standard atmospheric conditions. Subsequently, the scaffolds were placed within a vacuum dessicator to ensure complete evaporation of the solvent. Finally, the porogen was leached from the scaffolds via immersion in deionized water.

### Example 2

Three-dimensional microspheres of both PDL and PLLA/PLC were fabricated via a single emulsion, solvent evaporation technique, according “Adipocyte Response to Injectable Breast Tissue Engineering Scaffolds,” A N Cavin, S E Ellis, K J L Burg, Transactions of the 30^{th} Annual Meeting of the Society for Biomaterials, Memphis, Tenn. 2005, incorporated by reference herein. Briefly, the polymer of interest was dissolved in dichloromethane to make a 30% solution (weight of polymer to volume of solvent). Microspheres were then made with and without linoleic acid according to the following.

Linoleic acid (C18:2) was added to the solution in the amount required to make the final solution 16.67% weight linoleic acid to volume of solvent.

The resulting solution (either with or without linoleic acid) was then poured into a 1% poly(vinyl alcohol) solution, and the microspheres were extracted after at least 12 hours of mechanical agitation. The microspheres were collected by filtration, washed twice with 95% ethanol, dried, and stored in a desiccator until use.

The encapsulation efficiency of linoleic acid was found to be 39.6±1.8%, and only 4.3% of the linoleic acid was recovered after brief washing with 100% ethanol, indicating that the majority of the linoleic acid was entrapped within the cores of the microspheres. Without wishing to be bound by theory, the relatively low encapsulation efficiency is likely due to the long agitation time and the high initial loading of the linoleic acid. Despite the encapsulation efficiency, the loading of the microspheres was relatively high: at 179.95±7.14 mg linoleic acid per gram PDL.

The PDL microspheres encapsulating linoleic acid were evaluated via scanning electron microscopy (SEM) using a Philips XL series ESEM. The samples were mounted to aluminum stubs with double-sided carbon tape and sputter-coated with gold, using a Cressington Sputtercoater 108auto), prior to viewing. Micrographs were analyzed using ImageJ software from NIH. The mean diameter was determined to be 355 μm, with over 87% of the microspheres having diameters between 250 μm and 500 μm. The microspheres were spherical with a slightly roughened surface.

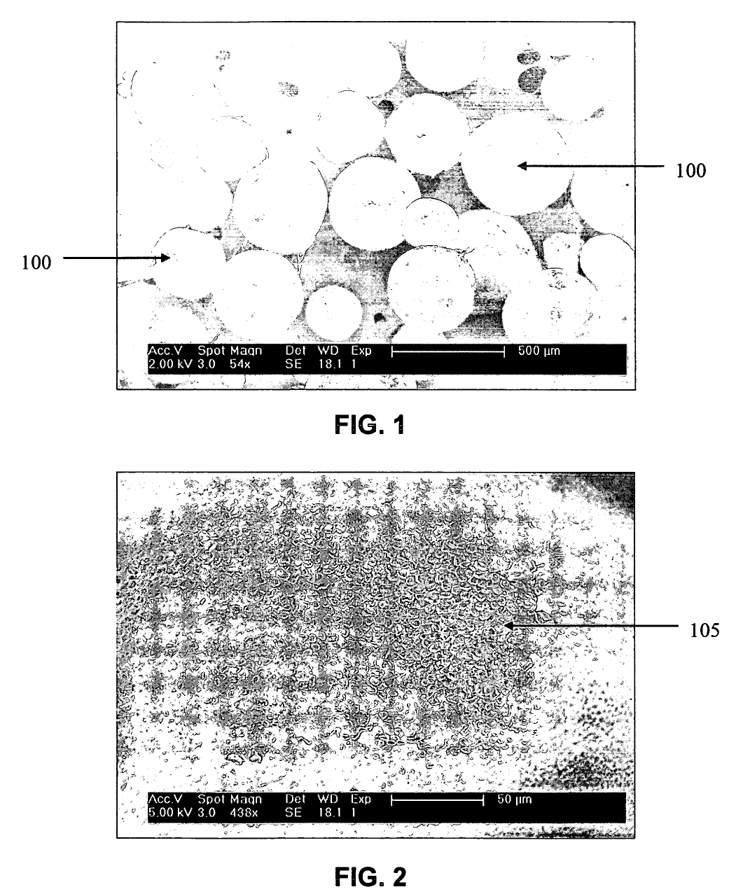

Referring now to FIGS. 1 and 2, the microspheres 100 are shown having an exterior surface 105. Exterior surface 105 is defined by the protective coating comprising the PDL polymer.

In vitro Rumen Incubations

A fluid inoculum containing a mixed microbial population was collected from the rumen of a fistulated Holstein cow housed at LaMaster Dairy Farm, Clemson, S.C. The scaffolds (from Example 1) and microspheres (from Example 2) were exposed to the fluid inoculum to determine their response to the mixed microbial population and their potential for surviving the mixed microbial environment. The samples were each incubated in the ruminal inoculum for 24 hours under physiological conditions: a constant temperature of 39° C., a pH maintained between 6 and 6.5, and anaerobic. Once the 24-hour incubation was complete, the samples were vacuum dried and weighed to determine a percentage weight change. Gas chromatography was employed for quantification of fatty acids (from Example 2), with a direct methylation procedure in methanolic HCl being used to prepare methyl esters of fatty acids for the GC analysis.

The results, in Table 1, show minimal losses of the PDL and PLLA/PLC over the 24 hour incubation period, indicating that both PDL and PLLA/PLC are capable of substantially surviving the microbial population.

TABLE 1

| | Average % Weight |
|---|---|
| Sample | Change |
| PDL Scaffolds | −1.08% +/− 1.15% |
| PDL Microspheres | −11.48% +/− 0.49% |
| PLLA/PLC Scaffolds | 0.50% +/− 10.40% |
| PLLA/PLC | −13.29% +/− 0.38% |
| Microspheres (w/o | |
| linoleic acid) | |
| PDL Microspheres (with | −6.24 +/− 2.32% |
| Linoleic Acid) | |

The PDL microspheres with linoleic acid were also tested for linoleic acid retention after the 24-hour ruminal incubation by a fatty acid analysis using gas chromatography, with results shown in Table 2. The high retention of linoleic acid corresponds well with the limited amount of weight degradation observed in the PDL microspheres with linoleic acid.

TABLE 2

| | Concentration | Concentration | |
|---|---|---|---|
| | (mg UFA/g | (mg UFA/g | |
| | microspheres) | microspheres) | |
| | Incubation | Incubation | |
| Fatty Acid | Time = 0 h | Time = 24 h | % Retention |
| Linoleic | 147.93 +/− 7.05 | 123.99 +/− 7.81 | 83.80% +/− 2.68% |
| Acid | | | |
| Total UFA | 219.98 +/− 8.06 | 194.00 +/− 9.92 | 88.16% +/− 1.42% |

In vitro Abomasum Incubations

Subsequent to the in vitro ruminal incubation, the microspheres of PDL encapsulating linoleic acid were exposed to a simulated ruminant abomasum environment. The simulated ruminant abomasum environment was created through a solution of pepsin and an HCl buffer system used to simulate the acidic conditions of the abomasum of a ruminant. The microspheres of PDL encapsulating linoleic acid were exposed to this environment for 24 hours, with the results shown in Table 3.

TABLE 3

| | Average % |
|---|---|
| Sample | weight change |
| PDL microspheres with linoleic acid | −8.38 +/− 0.67 |
| PDL microspheres w/o linoleic acid | −3.40 +/− 0.19 |

Interestingly, the PDL microspheres encapsulating linoleic acid showed better degradation in the acidic conditions, when compared to the PDL microspheres without any linoleic acid. Without wishing to be bound by theory, it is believed that the presence of enzymes in the abomasum of a ruminant will further increase the degradation rate of the microspheres. Additionally, pancreatic digestive enzymes in the intestine may also facilitate degradation of the polymers.

The amount of PDL degradation throughout the process is shown using GPC and DSC analysis in Table 4.

TABLE 4

| Characteristic | Amorphous Raw | | | Post simulated |
|---|---|---|---|---|
| (units) | Value | Sphere | Post ruminal fluid | abomasum fluid |
| MW (Daltons) | 23060.67 +/− 864.05 | 18743.7 +/− 954.71 | 20122.7 +/− 417.4 | 18919 +/− 2571.26 |
| Polydispersity | 8.21 +/− 2.02 | 1.62 +/− 0.17 | 1.55 +/− 0.01 | 1.61 +/− 0.11 |
| Tg (° C.) | 59.57 +/− 0.11 | 51.41 +/− 1.08 | 49.97 +/− 0.27 | 49.86 +/− 0.68 |

As shown in Table 4, the PDL polymer exhibited low variability throughout the testing, indicating that the harsh microbial environment of the rumen does not fundamentally alter the polymers' characteristics. As such, the degradation and release rates should be predictable to one of ordinary skill in the art by varying properties of the polymers.

1. A feed supplement for protecting unsaturated fatty acid from hydrogenation, the feed supplement comprising:
an unsaturated fatty acid; and
a protective coating encapsulating said unsaturated fatty acid, said protective coating comprising a biocompatible polymer, wherein at least 50% of said unsaturated fatty acid encapsulated by said protective coating remains unhydrogenated after the feed supplement is exposed to ruminal fluid for at least about 24 hours.

2. A feed supplement as in claim 1, wherein said biocompatible polymer is biodegradable through hydrolysis.

3. A feed supplement as in claim 1, wherein said biocompatible polymer is biodegradable through enzymatic hydrolysis.

4. A feed supplement as in claim 1, wherein said biocompatible polymer is a biocompatible aliphatic polyester.

5. A feed supplement as in claim 4, wherein said biocompatible polymer is a polyhydroxyacid.

6. A feed supplement as in claim 5, wherein said biocompatible polymer is a lactide-based polymer.

7. A feed supplement as in claim 1, wherein said synthetic polymer is selected from the group consisting of polylactide, polylactide-co-caprolactone, aliphatic polyesters, poly(amino acids), copoly(ether-esters), polyalkylene oxalates, polyamides, poly(iminocarbonates), polyacetals, polycyanoacrylates, degradable polyurethanes, polyorthoesters, polyoxaesters, polyamidoesters, polyoxaesters comprising amine groups, poly(anhydrides), polyphosphazenes, polysaccharides, polyacrylates, and poly(vinyl amines).

8. A feed supplement as in claim 1, wherein said biocompatible polymer is a natural polymer.

9. A feed supplement as in claim 8, wherein said natural polymer is selected from the group consisting of alginate, gelatin, proteins, albumin, synthetic polyamino acids, prolaminescollagen, polysaccharide, gelatin, fibrin, hyaluronic acid, agar, agarose, gum arabic, fibronectin, laminin, glycosaminoglycan, and polyvinyl alcohol.

10. A feed supplement as in claim 1, wherein said protective coating degrades in an acidic environment having a pH of less than about 3.

11. A feed supplement as in claim 1, wherein said protective coating comprises at least two biocompatible polymers.

12. A feed supplement as in claim 1, wherein said protective coating comprises a first layer and a second layer, wherein said first layer comprises a first biocompatible polymer and said second layer comprises a second biocompatible polymer, wherein said first biocompatible polymer is different than said second biocompatible polymer.

13. A feed supplement as in claim 1, wherein said biocompatible polymer has a crystallinity of from 0% to about 40%.

14. A feed supplement for protecting unsaturated fatty acid from hydrogenation, the feed supplement comprising:
an unsaturated fatty acid; and
a protective coating encapsulating said unsaturated fatty acid, wherein said protective coating comprises a biocompatible aliphatic polyester polymer that is biodegradable through hydrolysis.

15. A feed supplement as in claim 14, wherein said biocompatible aliphatic polyester polymer is a lactide-based polymer.

16. A feed supplement as in claim 15, wherein said lactide-based polymer is selected from the group consisting of polylactides and polylactide-co-caprolactones.

17. A feed supplement as in claim 14, said protective coating degrades in an aqueous environment having a pH of less than about 6.

18. A feed supplement as in claim 14, wherein at least 50% of said unsaturated fatty acid encapsulated by said protective coating remains unhydrogenated after the feed supplement is exposed to ruminal fluid for at least about 24 hours.

19. A method preparing a feed supplement for ruminants, the method comprising
encapsulating unsaturated fatty acid with a protective coating, wherein the protective coating comprises a biocompatible aliphatic polyester polymer, wherein at least 50% of said unsaturated fatty acid encapsulated by said protective coating remains unhydrogenated after the feed supplement is exposed to a ruminal fluid for at least about 24 hours.

20. A method as in claim 12, wherein said biocompatible aliphatic polyester polymer is a lactide-based polymer.
